(19) United States
(12) Patent Application Publication
(10) Pub. No.: US 2026/0265821 A1
(43) Pub. Date: Sep. 10, 2026

(54) METHODS FOR DETERMINING MRNA CAPPING EFFICIENCY

(71) Applicant: Hongene Biotech Corporation, Union City, CA (US)

(72) Inventors: Nien-Ching Han, Union City, CA (US); Gaomai Yang, Union City, CA (US); David Yu, Union City, CA (US)

(21) Appl. No.: 19/557,001

(22) Filed: Mar. 4, 2026

Related U.S. Application Data
(60) Provisional application No. 63/767,262, filed on Mar. 5, 2025.

(51) Int. Cl. C12Q 1/6876

(52) U.S. Cl. CPC C12Q 1/6876

(57) ABSTRACT

Aspects of the present disclosure generally relate to sensitive and accurate methods and compositions for determining mRNA capping efficiency. More specifically, aspects of the present disclosure relate to the use of malachite green to detect in vitro transcription mRNA capping efficiency.

### INCORPORATION BY REFERENCE TO PRIORITY APPLICATION

The present application claims the benefit of priority to U.S. provisional patent application No. 63/767,262, filed Mar. 5, 2025, the entirety of which is incorporated by reference herein.

### FIELD

Aspects of the present disclosure generally relate to methods for determining messenger RNA (mRNA) capping efficiency.

### BACKGROUND

The five-prime (5′) cap is a specially altered nucleotide on the 5′ end of some mRNA. The 5′ cap binds to the translation initiation complex and allows translation initiation to occur. The 5′ cap also defends the mRNA from 5′ exonucleases, resulting in increased mRNA stability. For the manufacture of RNA therapeutics, in vitro synthesis methods are preferred. During in vitro synthesis capping structures can be introduced either post-transcriptionally (enzymatically) or co-transcriptionally. Post-transcription, or enzymatic, capping can be efficient but requires multiple purification steps. In contrast, co-transcriptional capping requires only a single purification step. However, co-transcriptional capping utilizes expensive cap analogs and co-transcriptional capping efficiency varies with the cap analog used. Capped mRNAs have found broad application for in vitro protein synthesis as well as for obtaining expression of exogenous mRNAs in living cells. Given the simplicity of co-transcriptional capping, cap analogs are a very popular product on the market. Accordingly, there is a need for a rapid and efficient high-throughput method to screen cap analogs.

Malachite green (MG) phosphate assay was reported by Baykov AA, et al., A malachite green procedure for orthophosphate determination and its use in alkaline phosphatase-based enzyme immunoassay, Anal Biochem. 1988 June;171(2):266-70. Methods of measuring post-transcription capping efficiency of mRNAs using Malachite green phosphate assay have been reported by Gunawardana D, et al., Efficient preparation and properties of mRNAs containing a fluorescent cap analog: Anthraniloyl-m(7)GpppG, Translation (Austin) 2015 February 2;3(1):e988538; Ying Tu, et al., Capped or uncapped? Techniques to assess the quality of mRNA molecules, Current Opinion in Systems Biology, Volume 37, 2024, p.100503. However, the speed and accuracy and scalability of these methods when using co-transcriptionally capped mRNA are limited. There is therefore a need for improved methods of measuring mRNA capping efficiently that can offer results that are fast, accurate and scalable.

### SUMMARY

Some embodiments provided herein are described by way of the following numbered embodiments and also provided as possible combinations or overlapping embodiments.

Some embodiments provided herein relate to a method of determining mRNA capping efficiency (e.g., co-transcriptional capping efficiency) in a test sample. In some embodiments, the method of determining mRNA capping efficiency in a test sample comprises: treating a test sample and one or more control samples with an alkaline phosphatase (AP); adding a phosphate detection agent to the test sample and the one or more control samples treated with the alkaline phosphatase; detecting the amount of free phosphate groups in the test sample and the one or more control samples; determining the ratio of free phosphate groups in the test sample to the free phosphate groups in the one or more control samples; and determining the mRNA capping efficiency in the test sample. In some embodiments, the test sample comprises capped and uncapped mRNAs, and the one or more control samples each comprise a known ratio of capped to uncapped mRNA. In some embodiments, at least one control sample comprises 100% uncapped mRNA. In some embodiments, at least one control sample comprises a known ratio of capped to uncapped mRNA. In some embodiments, the mRNA capping efficiency in the test sample is equal to 1 minus the percentage of uncapped mRNA. In some embodiments, the percentage of uncapped mRNA is determined by the ratio of the amount of free phosphate groups in the test sample to the amount of free phosphate groups in the one or more control samples, after accounting for any background phosphate signal. In some embodiments, determining mRNA capping efficiency comprises determining the amount of free phosphate in one or more test samples. In some embodiments, determining the amount of free phosphate in one or more test samples comprises measuring the optical density of the one or more test samples and determining the concentration of free phosphate using a standard phosphate curve. In some embodiments, determining the free phosphate concentration further comprises measuring alkaline phosphatase independent background phosphate, mRNA independent background phosphate, and/or buffer phosphate concentrations. In some embodiments, one or more of the alkaline phosphatase independent background phosphate, mRNA independent background phosphate, and/or buffer phosphate concentrations are subtracted from the measured free phosphate concentration of one or more test samples. In some embodiments, once the concentration of free phosphate in one or more test and control samples is determined, the ratio between free phosphate the one or more test and control samples is used to determine the percentage of uncapped mRNA in the one or more test samples. For example, in some embodiments, the percentage of uncapped mRNA in the one or more test samples equals 100 multiplied by the quotient of the concentration of free phosphate the one or more test samples divided by the concentration of free phosphate in one or more control samples. In some embodiments, capping efficiency is then calculated as equal to 100 multiplied by the result of 1 minus the percentage of uncapped mRNA in the one or more test samples. In some embodiments, the capped mRNA in the test sample is prepared by in vitro transcription with a cap analog. In some embodiments, the cap analog is selected from m⁷GpppA(_{2′-OMe})pG, m⁷GpppA(_{2′-oEt})pG, m⁷GpppA(_{2′-OMe})pU, m⁷GpppA(_{2′-OEt})pU, m⁷G(_{3′-OMe})pppA(_{2′-OMe})pG, m⁷G(_{3′OMe})pppA(_{2′-OMe})pU, m⁷G(_{3′-OMe})pppA(_{2′-OEt})pG, m⁷G(_{3′OMe})pppA(_{2′-oEt})pU or m⁷G(_{3′OMe})pppG, or combinations thereof. In some embodiments, the concentration alkaline phosphatase is from about 0.01 U/μL to about 5 U/μL. In some embodiments, the alkaline phosphatase comprises TAB5. In some embodiments, the alkaline phosphatase comprises CIP. In some embodiments, the treating of the test sample and the one or more control samples with an alkaline phosphatase comprises incubating the test sample and the one or more control samples in the presence of the alkaline phosphatase for a period of time to allow for the release of all three 5′ terminal phosphate groups from the uncapped mRNA. In some embodiments, the incubating is carried out at room temperature. In some embodiments, the incubating is carried out at about 37° C. temperature. In some embodiments, the incubating is carried out for about 10 minutes to about 1 hour. In some embodiments, the incubating is carried out for about 30 minutes. In some embodiments, treating the test sample and the one or more control samples with the alkaline phosphatase is in the presence of a RNase inhibitor. In some embodiments, the phosphate detection agent comprises Malachite Green. In some embodiments, adding the phosphate detection agent quenches the alkaline phosphatase reaction with the test sample and the one or more control samples. In some embodiments, the method further comprises centrifuging the test sample and the one or more control samples after adding the phosphate detection agent. In some embodiments, detecting the amount of free phosphate groups in the test sample and the one or more control samples comprises measuring the optical density of the test sample and the one or more control samples. In some embodiments, the optical density of the test sample and the one or more control samples is measured at about 600 nm to about 660 nm, or about 620 nm. In some embodiments, the amount of mRNA in the test sample is from about 0.5 μg/μL to about 10 μg/μL. In some embodiments, the amount of free phosphate groups detectable by the phosphate detection agent is from about 0.01 to about 100 μM, or from about 0.02 μM to about 40 μM. In some embodiments, at least one control sample comprises no mRNAs. In some embodiments, at least one control sample is not treated with the alkaline phosphatase. In some embodiments, the capping efficiency of the test sample determined by the method is within 10% of the caping efficiency of the test sample as measured by LC-MS. In some embodiments, the method further comprises heating the test sample and one or more control samples prior to treating the test sample and one or more control samples with an alkaline phosphatase. In some embodiments, the test sample and one or more control samples are heated to between about 65-95° C. In some embodiments, the test sample and one or more control samples are heated for between about 2-10 minutes. In some embodiments, the test sample and one or more control samples are heated to between about 65-95° C. for between about 2-10 minutes.

### BRIEF DESCRIPTION OF THE DRAWINGS

FIG. 1 is an illustration outlining a method for determining mRNA capping efficiency according to certain embodiments of the present disclosure.

FIG. 2A is a graph depicting quantification of phosphate release from 1 μg/μL uncapped mRNA incubated with 0.25 U/μL alkaline phosphatase at 4° C. over time.

FIG. 2B is a graph depicting some embodiments of quantification of phosphate release from 1 μg/μL uncapped mRNA incubated without any alkaline phosphatase at 4° C. over time.

FIG. 3A is a graph depicting quantification of phosphate release from 1 μg/μL uncapped mRNA at 37° C. with and without alkaline phosphatase over time according to certain embodiments of the present disclosure.

FIG. 3B is a graph depicting quantification of alkaline phosphatase (AP) dependent phosphate release from 1 μg/μL uncapped mRNA at 37° C. subtracting no AP background in FIG. 3A.

FIG. 3C is a graph depicting quantification of AP dependent phosphate release from 1 μg/μL denatured eGFP mRNA at 4° C.

FIG. 3D is a graph depicting quantification of AP dependent phosphate release from 1 μg/μL denatured eGFP at 25° C.

FIG. 4A is a graph depicting quantification of AP dependent phosphate release from 0.5 μg/μL Fluc (N1meψ) according to certain embodiments of the present disclosure.

FIG. 4B is a graph depicting some embodiments of quantification of AP dependent phosphate release from 0.5 μg/μL unmodified Fluc according to certain embodiments of the present disclosure.

FIG. 5A is the chemical structure of a HiXCap™ E1 cap analog.

FIG. 5B is a graph depicting quantification of AP dependent phosphate (Pi) release over time from Fluc mRNA with or without a HiXCap™ E1 cap.

FIG. 5C is the chemical structure of a HiXCap™ E2 cap analog.

FIG. 5D is a graph depicting quantification of AP dependent phosphate (Pi) release over time from Fluc mRNA with or without a HiXCap™ E2 cap.

FIG. 6 is a graph showing quantification of AP dependent phosphate (Pi) release over time from an eGFP mRNA mixture with an increasing percentage of ARCA capped mRNA according to certain embodiments of the present disclosure.

FIG. 7 is a graph depicting quantification of AP dependent phosphate (Pi) release from an uncapped/GAG capped Cas9 mRNA mixture according to certain embodiments of the present disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless specified otherwise, all technical and scientific terms used herein have the same meaning as is commonly understood by one of skill in the art to which the claimed subject matter belongs when read in light of the specification. It is to be understood that the foregoing general description and the following detailed description are exemplary and explanatory only and are not restrictive of any subject matter claimed.

The section headings used herein are for organizational purposes only and are not to be construed as limiting the subject matter described.

### Terminology

The articles “a” and “an” have its plain and ordinary meaning as read in light of the specification and refer to one or to more than one (for example, at least one) of the grammatical object of the article. By way of example, “an element” means one element or more than one element.

Throughout this specification, unless the context requires otherwise, the words “comprise,” “comprises,” and “comprising” has their plain and ordinary meaning as read in light of the specification and will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

### Embodiments of the Disclosure

Provided herein are methods of determining 5′ mRNA capping efficiency. The methods can include treating a test sample and one or more control samples with an alkaline phosphatase. The test sample can include a known or an unknown ratio of capped to uncapped mRNA. In some embodiments, the capped mRNA in the test sample is prepared by in vitro transcription with a cap. In some embodiments, the cap is a synthetic cap analog. In some embodiments, more than one test sample is provided. The one or more control samples each comprise a known ratio of capped to uncapped mRNA. In some embodiments, the alkaline phosphatase comprises Tab5 or CIP alkaline phosphatase. The alkaline phosphatase treatment releases the 5′ phosphate(s) from only uncapped mRNA. A phosphate detection agent is then added to the test sample and the one or more control samples treated with the alkaline phosphatase. In some embodiments, contacting the control and/or test sample with the phosphate detection agent quenches the phosphatase reaction. The phosphate detection agent can include molybdate, for example, but not limited to sodium molybdate and/or ammonium molybdate, and a dye suitable for recognition of the product formed between the free phosphate and the molybdate, i.e., phosphomolybdate. For example, in the methods provided herein, the dye can be, but is not limited to, malachite green. The amount of free phosphate groups in the test sample and the one or more control samples is then detected and compared to the amount of free phosphate in the test sample to determine the capping efficiency. The methods provided herein may further include preparing the phosphate detection agent. In some embodiments, preparing the phosphate detection agent can include dissolving a phosphomolybdate recognizing dye in an acidic solution, followed by the addition of molybdate. In some embodiments, the molybdate is added just prior to contacting the test and/or control sample.

The phosphate detection agent can include ammonium molybdate and a dye. Suitable dyes for recognition of phosphomolybdate, for example, but not limited to, malachite green, are known in the art. The amount of free phosphate in the control sample and test sample can then be determined, with the ratio of free phosphate between the control samples and test sample indicating the mRNA capping efficiency. In some embodiments, the amount of free phosphate is determined by measuring the optical density of the sample.

mRNA cap analogs are known in the art. Any suitable mRNA cap can be used in the methods provided herein. For example, the cap can be a methylated cap, for example, a monomethylated or tri-methylated cap; an unmethylated cap; an anti-reverse cap analog (ARCA), or an A cap. In some embodiments, the cap can include, but is not limited to, GP₃7, 3′-O-Me-m7G(5′) ppp(5′)G, m⁷GP₃G, m⁷GpppGmpG, m⁷GpppGmpG ammonium, m⁷GpppAmpG ammonium, m₂^{2.2-7}GP₃G, m₂^{7,3′-O}GP₃G, GAG, M6, GAG3OMe or 3′OMeGA2′-OETG, AP₃G, m⁷G(5′)ppp(5′)A, G(5′)ppp(5′)A, m⁷Gpppm⁶AmpG, uracil-m⁷GpppAmpG ammonium, m⁷G(5′)ppp(5′)(2′OMeA)pU, m⁷G(5′)ppp(5′)(2′OMeA)pG, m⁷(3′OMeG)(5′)ppp(5′)(2′OMeA)pG, N7-Methyl-guanosine-5′-triphosphate-5′-adenosine diammonium, N7-Methyl-guanosine-5′-triphosphate-5′-adenosine, m⁷GpppCpG, m⁷GpppApG, m⁷GpppUpG, m⁷GpppAmpG, m⁷Gpppm⁶AmpG ammonium, M⁷G(3′-OMe-5′)pppA(2′-OMe), m⁷GpppCmpG, 3′Ome-m⁷GpppAmpG, m⁷GpppGpG, m⁷GpppUmpG, m⁷-3′_{OMe}-GDP, anthraniloyl-m⁷GpppG, m⁷G_{3′O-Me}pppA_{2′O-ethyl}pG, m⁷GpppA_{2′O-ethyl}pG, m⁷G_{3′O-Me}ppp^{m6}A_{2′O-ethyl}pG, m⁷GpppA_{2′O-methoxyethyl}pApG, m⁷G_{3′O-Me}pppA_{2′O-methoxyethyl}pG, m⁷G_{3′O-Me}PPP^{m6}A_{2′O-methoxyethyl}pG, m⁷Gppp^{m6}A_{2′O-Me}pA_{2′OMe}pG, m⁷G_{3′O-me}pppA_{2′O-ethyl}pApG, m⁷G_{3′O-Me}ppp^{m6}A_{2′O-ethyl}pApG, m⁷GpppA_{2′O-me}pA_{2′O-Me}pG, m⁷GpppA_{2′O-Me}pG_{2′O-me}pG, m⁷GpppA_{2′O-Me}pU_{2′O-me}pG, m⁷G_{3′O-Me}pppA_{2′O-me}pA_{2′O-me}pG, m⁷G_{3′O-Me}pppA_{2′O-Me}pG_{2′O-me}pG, m⁷G_{3′O-Me}pppA_{2′O-me}pU_{2′O-me}pG, m⁷Gppp^{m6}A_{2′O-Me}pA_{2′O-Me}pG, m⁷GpppA_{2′O-Me}p^{m6}A_{2′O-Me}pG, m⁷Gppp^{m6}A_{2′O-Me}pG_{2′O-Me}pG, m⁷Gppp^{m6}A_{2′O-Me}pU_{2′O-Me}pG, m⁷G_{3′O-Me}ppp^{m6}A_{2′O-Me}pA_{2′O-me}pG, m⁷G_{3′O-Me}PPP^{m6}A_{2′O-Me}pG_{2′O-Me}pG, m⁷G_{3′O-Me}ppp^{m6}A_{2′O-Me}pU_{2′O-Me}pG, m⁷GpppA_{2′O-ethyl}pA_{2′O-Me}pG, m⁷GpppA_{2′O-ethyl}pG_{2′O-Me}pG, m⁷GpppA_{2′O-ethyl}pU_{2′O-Me}pG, m⁷G_{3′O-me}pppA_{2′O-ethyl}pA_{2′O-Me}pG, m⁷G_{3′O-Me}pppA_{2′O-ethyl}pG_{2′O-Me}pG, m⁷G_{3′O-Me}pppA_{2′O-ethyl}pU_{2′O-Me}pG, m⁷GpppA_{2′O-ethyl}p^{m6}A_{2′O-Me}pG, m⁷Gppp^{m6}A_{2′O-ethyl}pG_{2′O-Me}pG, m⁷Gppp^{m6}A_{2′O-ethyl}pU2′O-MepG, m⁷G_{3′O-Me}PPP^{m6}A_{2′O-ethyl}pA_{2′O-Me}pG, m⁷G_{3′O-Me}ppp^{m6}A_{2′O-ethyl}pG_{2′O-me}pG, or m⁷G_{3′O-Me}pppₘ₆A_{2′O-ethyl}pU_{2′O-Me}pG, and/or any combination thereof.

Multiple control samples may be provided in the methods disclosed herein. In some embodiments, a single control sample is provided. In some embodiments, 1, 2, 3, 4, 5, 6, 7 or 8 or more control samples are provided. In some embodiments, a number of control samples in a range defined by any two of the preceding values is provided. For example, in some embodiments, between 1-10, 1-7, 1-5, 1-3, 3-10, 3-7, 3-5, 5-10, 5-7, or 7-8 control samples are provided. In some embodiments, more than 10 control samples are provided.

The control samples may include a known ratio of capped to uncapped mRNA. The control sample can include about 0%, 1%, 2%, 3%, 4%, 5%, 6%, 7%, 8%, 9%, 10%, 20%, 25%, 30%, 40%, 50%, 60%, 70%, 75%, 80%, 90%, 91%, 92%, 93%, 94%, 95%, 96%, 97%, 98%, 99%, or 100% capped mRNA. In some embodiments, the control sample includes an amount of capped mRNA that is in a range defined by any two of the preceding values. For example, in some embodiments, the control sample comprises between about 0-100%, 0-95%, 0-90%, 0-75%, 0-50%, 0-25%, 0-10%, 0-7%, 0-5%, 0-3%, 0-1%, 1-100%, 1-95%, 1-90%, 1-75%, 1-50%, 1-25%, 1-10%, 1-7%, 1-5%, 1-3%, 3-100%, 3-95%, 3-90%, 3-75%, 3-50%, 3-25%, 3-10%, 3-7%, 3-5%, 5-100%, 5-95%, 5-90%, 5-75%, 5-50%, 5-25%, 5-10%, 5-7%, 7-100%, 7-95%, 7-90%, 7-75%, 7-50%, 7-25%, 7-10%, 10-100%, 10-95%, 10-90%, 10-75%, 10-50%, 10-20%, 20-100%, 20-95%, 20-90%, 20-75%, 20-50%, 50-100%, 50-95%, 50-90%, 50-75%, 75-100%, 75-95%, 75-90%, 90-100%, 90-95%, or 95-100% capped mRNA. The ratio of capped to uncapped mRNA in the control sample can be about 1:10, 1:9, 1:8, 1:7, 1:6, 1:5, 1:4, 1:3, 1:2, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10. In some embodiments, the ratio of capped to uncapped mRNA in the control sample can be a ration that is in a range defined by any two of the preceding values. For example, in some embodiments, the ratio of capped to uncapped mRNA in the control sample can be between about 10:1 and 1:10, 10:1 and 1:7, 10:1 and 1:5, 10:1 and 1:3, 10:1 and 1:1, 10:1 and 10:7, 10:1 and 10:5, 10:1 and 10:3, 7:1 and 1:10, 7:1 and 1:7, 7:1 and 1:5, 7:1 and 1:3, 7:1 and 1:1, 7:1 and 7:3, 7:1 and 7:5, 5:1 and 1:10, 5:1 and 1:7, 5:1 and 1:5, 5:1 and 1:3, 5:1 and 1:1, 5:1 and 3:1, 3:1 and 1:10, 3:1 and 1:7, 3:1 and 1:5, 3:1 and 1:3, 3:1 and 1:1, 1:1-1:10, 1:1-1:7, 1:1 and 1:5, 1:1 and 1:3, 1:1 and 1:7, 1:1 and 1:10, 1:3 and 1:10, 1:3 and 1:7, 1:3 and 1:5, 1:5 and 1:10, 1:5 and 1:7, and 1:7 and 1:10.

Multiple test samples may be provided in the methods disclosed herein. In some embodiments, a single control sample is provided. In some embodiments, 1, 2, 3, 4, 5, 6, 7, 8, 9, or 10, control samples are provided. In some embodiments, a number of control samples in a range defined by any two of the preceding values is provided. For example, in some embodiments, between 1-10, 1-7, 1-5, 1-3, 3-10, 3-7, 3-5, 5-10, 5-7, or 7-10 control samples are provided. In some embodiments, more than 10 test samples are provided.

In some embodiments one or more control and/or test samples may be heated prior to contacting the one or more test and/or control samples with alkaline phosphatase. In some embodiments, one or more of the one or more control and/or test samples is heated to at least about 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., or 95° C., or to a temperature that is in a range that is defined by any two of the preceding values. For example, in some embodiments, one or more test and/or control samples are heated to between about 60-95° C., 60-90° C., 60-85° C., 60-80° C., 60-75° C., 60-70° C., 60-65° C., 65-95° C., 65-90° C., 65-85° C., 65-80° C., 65-75° C., 65-70° C., 70-95° C., 70-90° C., 70-85° C., 70-80° C., 70-75° C., 75-95° C., 75-90° C., 75-85° C., 75-80° C., 80-95° C., 80-90° C., 80-85° C., 85-95° C., 85-90° C. or 90-95° C. In some embodiments, heating one or more control and/or test samples prior to contacting the one or more test and/or control samples with alkaline phosphatase reduces the secondary structure of the one or more control and/or test samples. In some embodiments, the one or more test and/or control samples are heated for at least about 2, 3, 4, 5, 6, 7, 8, 9, or 10 minutes or for an amount of time that is in a range that is defined by any two of the preceding values prior to contacting one or more of the one or more control and/or test samples with alkaline phosphatase. For example, in some embodiments, the one or more test and/or control samples are heated for between about 2-10, 2-7, 2-5, 2-3, 3-10, 3-7, 3-5, 5-10, 5-7 or 7-10 minutes prior to contacting one or more of the one or more control and/or test samples with alkaline phosphatase. In some embodiments, heating one or more control and/or test samples for between about 2-10 minutes prior to contacting the one or more test and/or control samples with alkaline phosphatase reduces the secondary structure of the one or more control and/or test samples. In some embodiments, In some embodiments, one or more of the one or more control and/or test samples is heated to at least about 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., or 95° C., or to a temperature that is in a range that is defined by any two of the preceding values for at least about, 3, 4, 5, 6, 7, 8, 9, or 10 minutes or for an amount of time that is in a range that is defined by any two of the preceding values prior to contacting one or more of the one or more control and/or test samples with alkaline phosphatase. For example, in some embodiments, one or more test and/or control samples are heated to between about 60-95° C., 60-90° C., 60-85° C., 60-80° C., 60-75° C., 60-70° C., 60-65° C., 65-95° C., 65-90° C., 65-85° C., 65-80° C., 65-75° C., 65-70° C., 70-95° C., 70-90° C., 70-85° C., 70-80° C., 70-75° C., 75-95° C., 75-90° C., 75-85° C., 75-80° C., 80-95° C., 80-90° C., 80-85° C., 85-95° C., 85-90° C. or 90-95° C. for between about 2-10, 2-7, 2-5, 2-3, 3-10, 3-7, 3-5, 5-10, 5-7 or 7-10 minutes prior to contacting one or more of the one or more control and/or test samples with alkaline phosphatase. In some embodiments, heating one or more control and/or test samples to between about 65-95° C. for between about 2-10 minutes prior to contacting the one or more test and/or control samples with alkaline phosphatase reduces the secondary structure of the one or more control and/or test samples.

In the methods provided herein, the control and test samples are contacted with an alkaline phosphatase. The alkaline phosphatase may be tissue specific or tissue non-specific alkaline phosphatase. In some embodiments, the alkaline phosphatase is a human alkaline phosphatase. In some embodiments, the alkaline phosphatase is calf intestinal alkaline phosphatase (CIP) or bacterial alkaline phosphatase. In some embodiments, the alkaline phosphatase is Tab5 alkaline phosphatase. The alkaline phosphatase may maintain activity at low temperature, allowing the some or all steps of the methods provided herein to be carried out on ice or at room temperature to avoid mRNA degradation during the heat incubation, which can release unwanted background phosphate.

Varying amounts of alkaline phosphatase may be used in the methods provided herein. For example, in some embodiments, about 0.01, 0.02, 0.025, 0.03, 0.04, 0.05, 0.06, 0.07, 0.075, 0.08, 0.09, 0.1, 0.2, 0.25, 0.3, 0.4, 0.5, 0.6, 0.7, 0.75, 0.8, 0.9, 1, 2, 2.5, 3, 4, 5, 6, 7, 7.5, 8, 9, or 10 U/μL, or an amount of alkaline phosphatase that is in a range defined by any two of the preceding values. For example, in some embodiments, between about 0.01-10 U/μL, 0.01-7.5 U/μL, 0.01-5 U/μL, 0.01-2.5 U/μL, 0.01-1 U/μL, 0.01-0.1 U/μL, 00.01-0.05 U/μL, 0.05-10 U/μL, 0.05-7.5 U/μL, 0.05-5 U/μL, 0.05-2.5 U/μL, 0.05-1 U/μL, 0.05-0.1 U/μL, 0.1-10 U/μL, 0.1-7.5 U/μL, 0.1-5 U/μL, 0.1-2.5 U/μL, 0.1-1 U/μL, 0.1-0.5 U/μL, 0.5-10 U/μL, 0.5-7.5 U/μL, 0.5-5 U/μL, 0.5-2.5 U/μL, 0.5-1 U/μL, 1-10 U/μL, 1-7.5 U/μL, 1-5 U/μL, 1-2.5 U/μL, 2.5-10 U/μL, 2.5-7.5 U/μL, 2.5-5 U/μL, 5-10 U/μL, 5-7.5 U/μL or 7.5-10 U/μL.

The alkaline phosphatase reaction is allowed to proceed for an amount of time that is long enough for free phosphate to be released from the uncapped mRNA in the control and/or test samples. In some embodiments, the alkaline phosphatase reaction is allowed to continue for about 1, 2, 3, 4, 5, 6, 7, 8, 9, 10, 15, 20, 25, 30, 35, 40, 45, 50, 55, or 60 minutes. In some embodiments, the alkaline phosphatase reaction is allowed to continue for about 30 minutes. In some embodiments, the alkaline phosphatase reaction is allowed to continue for a period of time that is in a range defined by any two of the preceding values. For example, in some embodiments, the alkaline phosphatase reaction is allowed to continue for between about 1-60, 1-45, 1-30, 1-15, 1-10, 1-5, 5-60, 5-45, 5-30, 5-15, 5-10, 10-60, 10-45, 10-30, 10-15, 15-60, 15-45, 15-30, 30-60, 30-45, or 45-60 minutes. In some embodiments, the alkaline phosphatase reaction is allowed to continue for between about 15-45 minutes.

The amount of released phosphate varies. In some embodiments, the amount of released phosphate is about 1×10⁻⁶, 5×10⁻⁶, 9×10⁻⁶, 1×10⁻⁵, 5×10⁻⁵, 9×10⁻⁵, 1×10⁻⁴, 5×10⁻⁴, 9×10⁻⁴, 1×10⁻³, 5×10⁻³, 9×10⁻³, 1×10⁻², 5×10⁻², 9×10⁻², 1×10⁻¹, 5×10⁻¹, 9×10⁻¹, and 1 μM. In some embodiments, the amount of released phosphate is an amount that is in a range defined by any two of the preceding values. For example, in some embodiments, the amount of released phosphate is between about 1×10⁻⁶ and 1 μM, 1×10⁻⁶ and 1×10⁻¹ μM, 1×10⁻⁶ and 1×10⁻² μM, 1×10⁻⁶ and 1×10⁻³ μM, 1×10⁻⁶ and 1×10⁻⁴ μM, 1×10⁻⁶ and 1×10⁻⁵ μM, 1×10⁻⁵ and 1 μM, 1×10⁻⁵ and 1×10⁻¹ μM, 1×10⁻⁵ and 1×10⁻² μM, 1×10⁻⁵ and 1×10⁻³ μM, 1×10⁻⁵ and 1×10⁻⁴ μM, 1×10⁻⁴ and 1 μM, 1×10⁻⁴ and 1×10⁻¹ μM, 1×10⁻⁴ and 1×10⁻² μM, 1×10⁻⁵⁴ and 1×10⁻³ μM, 1×10⁻³ and 1 μM, 1×10⁻³ and 1×10⁻¹ μM, 1×10⁻³ and 1×10⁻² μM, 1×10⁻² and 1 μM, 1×10⁻² and 1×10⁻¹ μM, and 1×10⁻¹ and 1 μM. In some embodiments, the amount of released phosphate is about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, or 100 μM. In some embodiments, the amount of released phosphate is an amount that is in a range defined by any two of the preceding values. For example, in some embodiments, the amount of released phosphate is between about 0.01-100 μM, 0.01-75 μM, 0.01-50 μM, 0.01-40 μM, 0.01-30 μM, 0.01-20 μM, 0.01-10 μM, 0.01-7 μM, 0.01-5 μM, 0.01-3 μM, 0.01-1 μM, 0.01-0.1 μM, 0.1-100 μM, 0.1-75 μM, 0.1-50 μM, 0.1-40 μM, 0.1-30 μM, 0.1-20 μM, 0.1-10 μM, 0.1-7 μM, 0.1-5 μM, 0.1-3 μM, 0.1-1 μM, 1-100 μM, 1-75 μM, 1-50 μM, 1-40 μM, 1-30 μM, 1-20 μM, 1-10 μM, 1-7 μM, 1-5 μM, 1-3 μM, 3-100 μM, 3-75 μM, 3-50 μM, 3-40 μM, 3- 30 μM, 3-20 μM, 3-10 μM, 3-7 μM, 7-100 μM, 7-75 μM, 7-50 μM, 7-40 μM, 7-30 μM, 7-20 μM, 7-10 μM, 10-100 μM, 10-75 μM, 10-50 μM, 10-40 μM, 10-30 μM, 10-20 μM, 20-100 μM, 20-75 μM, 20-50 μM, 20-40 μM, 20-30 μM, 30-100 μM, 30-75 μM, 30-50 μM, 30-40 μM, 40-100 μM, 40-75 μM, 40-50 μM, 50-100 μM, 50-75 μM, and 75-50 μM. In some embodiments, greater than 1000 μM of phosphate is released.

The steps of the methods provided herein may be carried out at the same temperature. Alternatively, different steps of the methods provided herein may be carried out at different temperatures. For example, in the methods provided herein, the one step may be performed on ice while another step may be performed at room temperature. In some embodiments, the alkaline phosphatase treatment may be performed at about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C. In some embodiments, the alkaline phosphatase reaction is performed at a temperature that is in a range defined by any two of the preceding values. For example, in some embodiments, the alkaline phosphatase reaction is carried out at between about 20-40° C., 20-35° C., 20-30° C., 20-25° C., 25-40° C., 25-35° C., 25-30° C., 30-40° C., 30-35° C., or 35-40° C.

In the methods provided herein, the control sample and the test sample are contacted with a phosphate detection agent. In some embodiments, the phosphate detection agent is acidic. In some embodiments, contacting the control and/or test sample with the acidic phosphate detection agent quenches the phosphatase reaction. The phosphate detection agent can include a dye which recognizes the product formed between the free phosphate (such as orthophosphate) and the molybdate, i.e., phosphomolybdate. Suitable dyes are known in the art. Any such dye, for example malachite green, can be used in the methods provided herein. In some embodiments, the dye is dissolved in an acidic solution or agent. For example, in some embodiments, the dye is dissolved in an acid, for example, but not limited to sulfuric acid, hydrochloric acid, trichloroacetic acid and/or another suitable acid or agent. The phosphate detection agent can include molybdate. In some embodiments, the molybdate can include sodium molybdate or ammonium molybdate, other possible molybdates include, but are not limited to one or more of aluminum molybdate, ammonium 6-molybdocobaltate, ammonium dimolybdate, ammonium heptamolybdate, ammonium molybdate, ammonium molybdate solution, ammonium molybdate tetrahydrate, ammonium octamolybdate, ammonium phosphomolybdate, ammonium phosphomolybdate hydrate, ammonium tetrathiomolybdate barium europium calcium molybdate, barium molybdate, bismuth iron molybdenum oxide, bismuth molybdate, bismuth molybdenum oxide, bismuth(III) molybdate, cadmium molybdate, calcium molybdate, calcium molybdenum oxide, cerium molybdenum oxide, cesium molybdate, cobalt molybdate, cobalt(II) molybdenum oxide hydrate, copper molybdate, copper molybdate sputtering target, iron(II) molybdate, lead molybdate, lithium molybdate, magnesium molybdate, manganese molybdate, nickel molybdate, nickel molybdate hydrate, phosphomolybdic acid hydrate, phosphomolybdic acid solution, potassium molybdate, rubidium molybdate, silver molybdate, sodium molybdate, sodium molybdate dihydrate, sodium molybdate anhydrous, sodium phosphomolybdate hydrate, strontium molybdate, thallium molybdate, zinc molybdate and/or any combination thereof. In some embodiments, the molybdate is added to the dye solution to form the phosphate detection agent just prior to contacting the control and/or test sample with the phosphate detection agent.

In the methods provided herein, the interaction between the phosphomolybdate and the dye results in a highly colored phosphomolybdate complex. For example, in some embodiments, the interaction between malachite green and phosphomolybdate under acidic conditions forms a green colored phosphomolybdate complex. The Malachite Green assay for free phosphate detection typically uses a reagent concentration of roughly 1% to 2% (e.g., 1.2% to 1.75% Malachite Green dye), often prepared in a sulfuric acid solution (e.g., 1.5 M-3M or 15%) and combined with a molybdate salt (e.g., ammonium molybdate).The amount of free phosphate in a sample can then be determined by measuring the optical density of the sample at an appropriate wavelength for recognition of the colored phosphomolybdate complex. In some embodiments, the optical density of the test sample and/or one or more control samples is measured in the supernatant of the sample. A skilled artisan will recognize that the appropriate wavelength will necessarily depend on the nature of the dye and the absorbance of the colored phosphomolybdate complex. For example, where the dye is malachite green, the optical density of the green phosphomolybdate complex can be measured at a wavelength of about 600 nm, 610 nm, 620 nm, 625 nm, 630 nm, 635 nm, 640 nm, 645 nm, 650 nm, 655 nm, or 660 nm. In some embodiments, the optical density of the green phosphomolybdate complex can be measured at a wavelength that is in a range defined by any two of the preceding values. For example, in some embodiments, the optical density of the green phosphomolybdate complex can be measured at a wavelength that is between about 600-660, 600-650, 600-640, 600-630, 600-610, 610-660, 610-650, 610-640, 610-630, 610-620, 620-660 nm, 620-650 nm, 620-640 nm, 620-630 nm, 630-660 nm, 630-650 nm, 630-640 nm, 640-660 nm, 640-650 nm, and 650-660 nm.

The optical density of a sample can be measured multiple times in methods provided herein. For example, in some embodiments, the optical density of a sample is measured 1, 2, 3, 4, 5, 6, 7, 8, 9, 10, 11, 12, 13, 14, or 15 times. In some embodiments, the optical density of a sample is measured a number of times that is in a range defined by any two of the preceding values. For example, in some embodiments, the sample is measured between 1-15, 1-10, 1-7, 1-5, 1-3, 3-15, 3-10, 3-7, 3-5, 5-15, 5-10, 5-7, 7-15, 7-10, or 10-15 times. In some embodiments, the optical density of the sample is measured about every 1, 2, 3, 4, 5, 6, 7, 8, 9, 10, 11, 12, 13, 14, 15, 20, 25, 30, 35, 40, 45, 50, 55, or 60 minutes. In some embodiments, the optical density of a sample is measured a number of times that is in a range defined by any two of the preceding values. For example, in some embodiments, the sample is measured between about every 1-60, 1-45, 1-30, 1-15, 1-10, 1-7, 1-5, 1-3, 3-60, 3-45, 3-30, 3-15, 3-10, 3-7, 3-5, 5-60, 5-45, 5-30, 5-15, 5-10, 5-7, 7-60, 7-45, 7-30, 7-15, 7-10, 10-60, 10-45, 10-30, 10-15, 15-60, 15-45, 15-30, 30-60, 30-45, or 45-60 minutes. In some embodiments, the optical density of a sample is measured until there is less than about a 0.1%, 0.2%, 0.3%, 0.4%, 0.5%, 0.6%, 0.7%, 0.8%, 0.9%, 1%, 1.25%, 1.5%, 1.75%, 2%, 2.25%, 2.5%, 2.75%, 3%, 3.25%, 3.5%, 3.75%, 4%, 4.25%, 4.5%, 4.75%, or 5% difference in two consecutive optical density readings of the same sample. In some embodiments, the optical density of a sample is measured until the difference in two consecutive optical density readings of the same sample is within a range defined by any two of the preceding values. For example, in some embodiments, the optical density of a sample is measured until there is less than between about a 0.1-5%, 0.1-4%, 0.1-3%, 0.1-2.5%, 0.1-2%, 0.1-1%, 0.1-0.9%, 0.1-0.5%, 0.5-5%, 0.5-4%, 0.5-3%, 0.5-2%, 0.5-1%, 0.5-0.9%, 1-5%, 1-4%, 1-3%, 1-2%, 2-5%, 2-4%, 2-3%, 3-5%, 3-4%, or 4-5% difference in two consecutive optical density readings of the same sample.

The percentage of uncapped mRNA can then be determined by comparing the optical density of a test sample to the optical density of one or more control samples. In some embodiments, the one or more control samples comprise 100% uncapped mRNA. In some embodiments, the one or more control samples comprise a known ratio of capped to uncapped mRNA. In some embodiments, the optical density of the control and/or test samples is converted to a concentration of free phosphate using a standard phosphate curve. In some embodiments, free phosphate background signal from enzyme, mRNA and/or buffer (phosphate produced that is not Alkaline phosphatase, mRNA and/or buffer dependent) is subtracted from the free phosphate concentration. In some embodiments, the percentage of uncapped mRNA is determined by comparing the amount of free phosphate groups in the test sample to the amount of free phosphate groups in one or more control samples, after accounting for any background signal. For example, in some embodiments, the percentage of uncapped mRNA in the one or more test samples equals 100 multiplied by the quotient of the concentration of free phosphate the one or more test samples divided by the concentration of free phosphate in one or more control samples. In some embodiments, the capping efficiency is then calculated according to Formula 1 as one minus the percentage of uncapped mRNA, i.e., 1 minus the ratio of the amount of free phosphate groups in one or more test samples to the amount of free phosphate groups in a control sample after accounting for any background signal. For example, in some embodiments, the one or more control samples comprise 0%, 1%, 2%, 3%, 4%, 5%, 6%, 7%, 8%, 9%, 10%, 20%, 25%, 30%, 40%, 50%, 60%, 75%, 80%, 90%, 91%, 92%, 93%, 94%, 95%, 96%, 97%, 98%, 99%, or 100% uncapped mRNA after accounting for any background signal, or comprise a percentage of uncapped mRNA that is in a range defined by any two of the preceding values. For example, in some embodiments, one or more control samples comprise between about 0-100%, 0-75%, 0-50%, 0-25%, 0-10%, 0-7%, 0-5%, 0-3%, 3-100%, 3-75%, 3-50%, 3-25%, 3-10%, 3-7%, 3-5%, 5-100%, 5-75%, 5-50%, 5-25%, 5-10%, 5-7%, 7-100%, 7-75%, 7-50%, 7-25%, 7-10%, 10-100%, 10-75%, 10-50%, 10-25%, 25-100%, 25-75%, 25-50%, 50-100%, 50-75% or 75-100% uncapped mRNA.

Capping Efficiency=100*(1−% uncapped mRNA) Formula 1

For example, in some embodiments, after subtracting background signals, the concentration of free phosphate for one or more uncapped samples, where cap analogs weren't added, is determined. In some embodiments, for one or more control samples, 100% of its mRNA is not capped. In some embodiments, one or more control samples comprises a known ratio of capped to uncapped mRNA. For one or more test samples, the uncapped % is calculated as equal to 100 multiplied by the quotient of the concentration of free phosphate the one or more test samples divided by the concentration of free phosphate in one or more control samples. Formula 1 is then applied to calculate capping efficiency, wherein the mRNA capping efficiency is equal to 1 minus X.

The methods provided herein are highly sensitive and accurate. For example, in some embodiments, the mRNA capping efficiency as determined by the methods provided herein are within about 0.1%, 0.2%, 0.3%, 0.4%, 0.5%, 0.6%, 0.7%, 0.8%, 0.9%, 1%, 1.25%, 1.5%, 1.75%, 2%, 2.25%, 2.5%, 2.75%, 3%, 3.25%, 3.5%, 3.75%, 4%, 4.25%, 4.5%, 4.75%, 5%, 5.25%, 5.5%, 5.75%, 6%, 6.25%, 6.5%, 6.75%, 7%, 7.25%, 7.5%, 7.75%, 8%, 8.25%, 8.5%, 8.75%, 9%, 9.25%, 9.5% 9.75%, 10%, 11%, 12%, 13%, 14%, or 15% of the capping efficiency of the same sample when measured by LC-MS. In some embodiments, the mRNA capping efficiency as determined by the methods provided herein are within a range that is defined by any two of the preceding values as compared to the capping efficiency of the same sample when measured by LC-MS. For example, in some embodiments, the mRNA capping efficiency as determined by the methods provided herein are within about 0.1-15%, 0.1-10%, 0.1-7%, 0.1-5%, 0.1-3%, 0.1-1%, 1-15%, 1-10%, 1-7%, 1-5%, 1-3%, 3-15%, 3-10%, 3-7%, 3-5%, 5-15%, 5-10%, 5-7%, 7-15%, 7-10%, or 10-15% of the capping efficiency of the same sample when measured by LC-MS.

### Compositions:

Provided herein are kits for performing the methods of the present disclosure. The kits may include a phosphomolybdate recognizing dye and one or more control samples. In some embodiments, the phosphomolybdate recognizing dye may be dissolved in an acidic solution. The dye may be any phosphomolybdate recognizing dye, for example, but not limited to, malachite green. The kits provided herein can include multiple control samples. For example, in some embodiments, the kits provided herein can include 1, 2, 3, 4, 5, 6, 7, 8, 9, or 10 control samples. The control samples can include a known ratio of capped to uncapped mRNA. For example, in some embodiments, the control samples can include 0%, 1%, 2%, 3%, 4%, 5%, 6%, 7%, 8%, 9%, 10%, 20%, 25%, 30%, 40%, 50%, 60%, 75%, 80%, 90%, 91%, 92%, 93%, 94%, 95%, 96%, 97%, 98%, 99%, or 100% capped mRNA. In some embodiments, the kits provided herein can include control samples having different ratios of capped to uncapped mRNA. In some embodiments, the kit further includes molybdate. In some embodiments, the molybdate is ammonium molybdate. In some embodiments, the kit further includes an alkaline phosphatase, an alkaline phosphatase buffer, RNase inhibitor, water, or any combination thereof.

### EXAMPLES

### Example 1

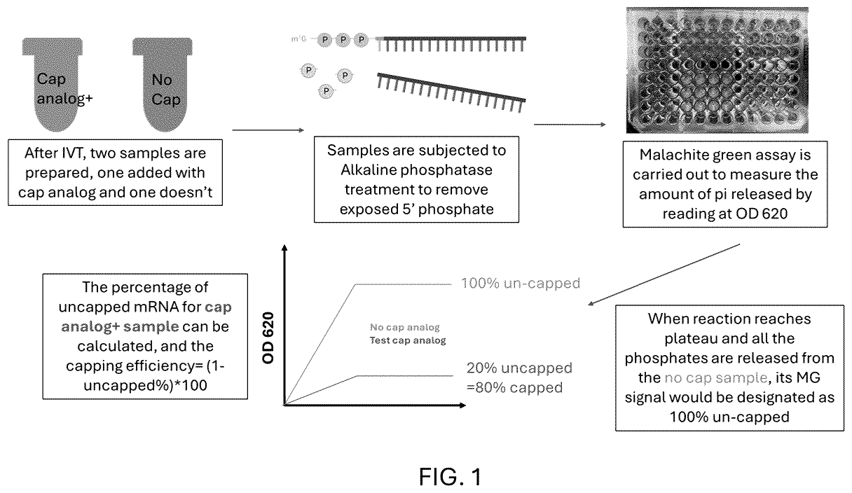

mRNA capping efficiency following in vitro transcription (IVT) was assessed using a malachite green phosphate detection assay (BioAssay Systems, Catalog No: POMG-25H). FIG. 1 is an illustration outlining the method. The reaction mixtures were prepared according to Tables 1-4.

Briefly, two IVT samples were prepared in parallel: one with a cap analog supplement to enable co-transcriptional capping and the other without cap analogs, serving as an uncapped control. After column purification, these two samples are named as capped and uncapped mRNA. mRNAs are heated at 70 degrees for 10 minutes then immediately put on ice. To measure the capping efficiency of co-transcriptional IVT, five reaction mixtures are set up: (1) buffer only; (2) 1 μg/μL capped mRNA with 0.25 U/μL alkaline phosphatase; (3) 1 μg/μL capped mRNA only; (4) 1 μg/μL uncapped mRNA with 0.25 U/μL alkaline phosphatase; (5) 1 μg/μL uncapped mRNA only.

TABLE 1
+mRNA + Enzyme

| | 1 RXN (μL) |
|---|---|
| 1 μg/μL heat denatured capped or uncapped mRNA | 50 |
| (2 μg/μL)* | |
| 1X alkaline phosphatase (“AP”) buffer (10X)* | 10 |
| 0.0625 U/μL alkaline phosphatase (2.5 U/μL)* | 10 |
| 1 U/μL RNase inhibitor (40 U/μL)* | 2.5 |
| H₂O | 27.5 |
| Total Volume | 100 |

*stock concentration

TABLE 2
+mRNA − Enzyme (Measuring AP independent
phosphate background (“A”))

| | 1 RXN (μL) |
|---|---|
| 1 μg/u heat denatured capped or uncapped mRNA | 50 |
| (2 μg/μL) | |
| 1X AP buffer (10X) | 10 |
| 0.25 U/μL AP (2.5 U/μL) | 0 |
| 1 U/μL RNase inhibitor (40 U/μL) | 2.5 |
| H₂O | 37.5 |
| Total Volume | 100 |

TABLE 3
−mRNA + Enzyme (Measuring the mRNA independent
pi background (“B”))

| | 1 RXN (μL) |
|---|---|
| 1X AP buffer (10X) | 10 |
| 0.25 U/μL AP (2.5 U/μL) | 10 |
| 1 U/μL RNase inhibitor (40 U/μL) | 2.5 |
| H₂O | 77.5 |
| Total Volume | 100 |

TABLE 4
−mRNA − Enzyme (Measuring AP and mRNA
independent pi background (“C”))

| | 1 RXN (μL) |
|---|---|
| 1X AP buffer (10X) | 10 |
| 1 U/μL RNase inhibitor (40 U/μL) | 2.5 |
| H₂O | 87.5 |
| Total Volume | 100 |

Before the reactions are quenched by the addition of malachite green phosphate detection agent, samples were diluted for 5 fold. This is to avoid the precipitation that occurs due to high concentrations of substrates.

The percentage of uncapped mRNA was then determined using a malachite green phosphate assay kit. 80 μL of each reaction mixture was added to a different well of a multi-well plate. L of malachite green phosphate detection agent was then added to each well, quenching the phosphatase reaction. The supernatants were used to read the OD620. The GD was fitted to a phosphate standard curve to determine the concentration (μM) of phosphates presented in different reaction mixtures. The results of the analysis are presented in Table S.

The phosphate concentration shown in Tables 5A-5E is multiplied by S to reflect the dilution factor. Table 5A depicts the measured phosphate concentration of the reactions shown in Tables 1-3. Table 5B shows the resulting phosphate concentration after subtracting background signals A and B. Background signal C may be measured and subtracted as necessary. The percentage of uncapped mRNA was then calculated. Table 5C shows the uncapped mRNA %. The percentage of capped mRNA was then calculated. Table 5D shows the capped mRNA %. The average capping efficiency of GAG capped mRNA was then calculated. Table 5E shows the CAG capping efficiency.

TABLE 5A

| | No cap | | | GAG Cap | | |
|---|---|---|---|---|---|---|
| Phosphate (μM) | Rep 1 | Rep 2 | Rep 3 | Rep 1 | Rep 2 | Rep 3 |
| +mRNA + AP | 78.78 | 83 | 83.28 | 11 | 12.38 | 11.63 |
| +mRNA − AP (A) | 5.25 | 5.5 | 5.9 | 3.88 | 4 | 3.93 |
| −mRNA + AP (B) | 1.35 | 1.18 | 1.58 | 1.35 | 1.18 | 1.58 |

TABLE 5B

| | No cap | | | GAG Cap | | |
|---|---|---|---|---|---|---|
| Phosphate (μM) | Rep 1 | Rep 2 | Rep 3 | Rep 1 | Rep 2 | Rep 3 |
| +mRNA + AP | 72.18 | 76.32 | 75.8 | 5.77 | 7.2 | 6.12 |

TABLE 5C

| | No cap | | | GAG Cap | | |
|---|---|---|---|---|---|---|
| Uncapped % | Rep 1 | Rep 2 | Rep 3 | Rep 1 | Rep 2 | Rep 3 |
| +mRNA + AP | 100 | 100 | 100 | 7.99 | 9.43 | 8.07 |

TABLE 5D

| | No cap | | | GAG Cap | | |
|---|---|---|---|---|---|---|
| capped % | Rep 1 | Rep 2 | Rep 3 | Rep 1 | Rep 2 | Rep 3 |
| +mRNA + AP | 0 | 0 | 0 | 92.01 | 90.57 | 91.93 |

TABLE 5E
GAG capping efficiency (%)
91.5 ± 0.8

The capping efficiency of GAG determined by MS is 99%, showing that the disclosed assay was highly sensitive and accurate.

### Example 2

Co-transcriptional IVT was carried out according to the procedure described in Example 1, and GAG capped and uncapped mRNAs are collected after LiCl purification. GAG capped and uncapped mRNAs (also referred to as “No Cap”) were then mixed at a 1:1 ratio. This sample is referred to as “50% Capped” in the following description. The reaction mixtures were prepared according to Tables 1-4 of Example 1. Both “50% Capped” and “No Cap” IVT mRNA were subjected to AP enzymatic reaction according to the recipe in Tables 1 and 2 of Example 1. In addition to Table 2 (+mRNA-Enzyme) which assesses non-enzymatic phosphate release, two negative controls were prepared in parallel as shown in Tables 3 and 4 of Example 1. The malachite green assay was then carried out according to Example 1.

In this example, the uncapped percentage of GAG mRNA was designated as “X”. The uncapped % was calculated as (X+100%)/2 because capped and uncapped mRNA are mixed at a 1:1 ratio. As such, the percentage of uncapped mRNA for “50% Uncapped” was equal to 50%+0.5×. Example experimental results are described below to facilitate the understanding of calculation. Table 7 shows the phosphate concentration after subtracting background A and B. Table 8 shows the percentage of uncapped mRNA in the samples after subtracting background.

TABLE 6

| Phosphate (μM) | No Cap | 50% Capped |
|---|---|---|
| +mRNA + AP | 80 | 45 |
| +mRNA − AP (A) | 5 | 3 |
| −mRNA + AP (B) | 1 | 1 |

TABLE 7

| Phosphate (μM) | No Cap | 50% Capped |
|---|---|---|
| +mRNA + AP | 74 | 41 |

TABLE 8

| Uncapped % | No Cap | 50% Capped |
|---|---|---|
| +mRNA + AP | 100 | 55 |

The equation 55=50+0.5(X) was then solved for X to determine the percentage of uncapped GAG IVT mRNA was 10%. The capping efficiency was then determined to be 90% according to Formula 1 as: 100%-10%=90%.

### Example 3: Incubation Study

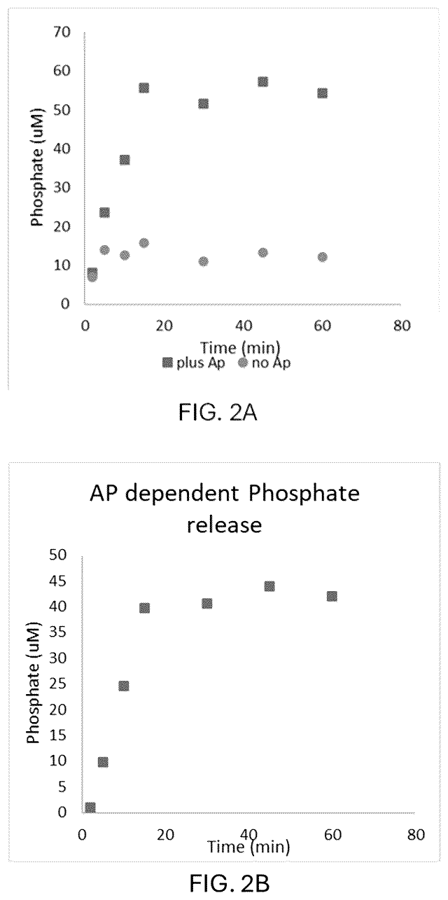

After co-transcriptional IVT and purification, GAG Cap and No Cap mRNAs were subjected to AP enzymatic treatment according to the recipes in Tables 1-2 of Example 1. Enzymatic treatment controls were also carried out according to Tables 3-4 of Example 1. AP treatment is conducted at 4 degrees Celsius for 30 minutes. After 30 minutes, the reaction was quenched by the addition of MG phosphate detection agent at a ratio of reaction mix to MG phosphate detection agent of 4:1. After 30 minutes of incubation, the samples were centrifuged at 3000g RCF for 10 min. Then the supernatant was taken, and the optical density of the mixture was read at OD620. The OD was fitted to a phosphate standard curve to determine the concentration (μM) of phosphates in the different reaction mixtures. The results of the analysis are presented in Tables 5A-5E. FIGS. 2A and 2B show time course results of 1 μg/μL uncapped mRNA incubated with or without 0.025 U/μL AP at 4° C. respectively. This result demonstrates that AP stays active at 4° C. and can reaches plateau in terms of digesting the unprotected 5′ triphosphates in as little as 15 minutes.

### Example 4: Time Course Study of Alkaline Phosphatase (AP) Dependent Phosphate Release at Different Temperatures

A time-course study was performed to characterize AP dependent release of free phosphate under different temperature conditions. Cas9 mRNA and eGFP mRNA were used as representative capped/uncapped mRNA templates.

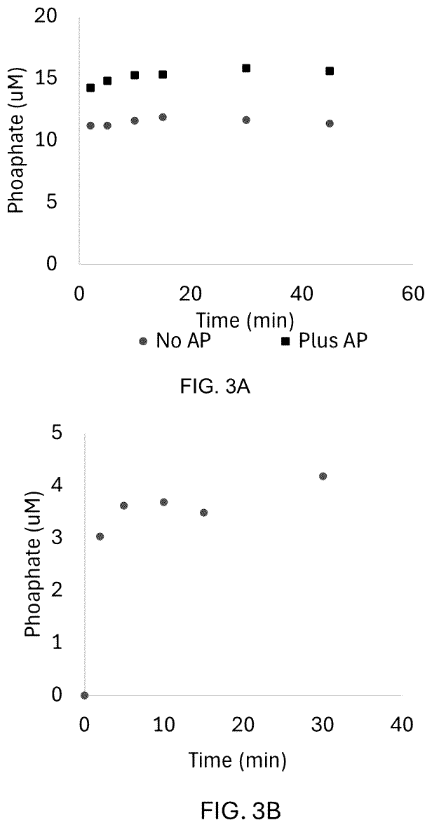

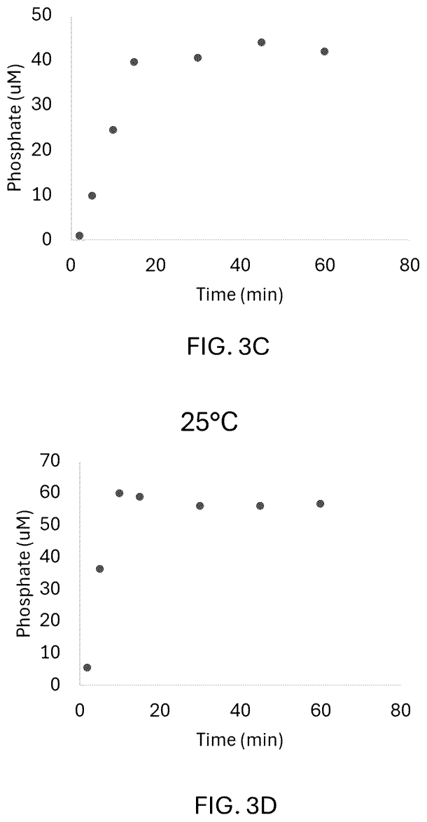

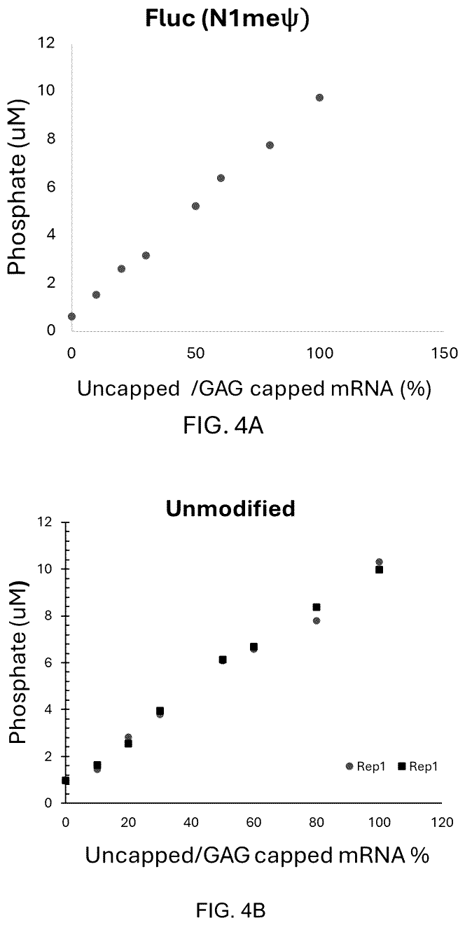

For Cas9 mRNA, samples were first heated at 70° C. for 5 minutes and immediately placed on ice to reduce secondary structure and expose 5′ terminal phosphates. Each reaction contained 1 μg/μL mRNA and 0.0625 U/μL AP. The reaction volume was made up to 100 μL using water. Reactions were incubated at 37° C. for 10 minutes, and aliquots were collected at 2 min, 5 minutes, 10 minutes, 15 minutes, 30 minutes, and 45 minutes. Reaction aliquots were diluted with water (2×) and quenched using malachite green quenching buffer. Quenched reactions were centrifuged for 10 min to remove precipitates, and supernatants were analyzed by absorbance measurement at 620 nm. FIG. 3A depicts quantification of phosphate release (μM) from 1 μg/μL uncapped Cas9 mRNA at 37° C. with and without AP. Background (no-enzyme) phosphate concentration was subtracted from the plus-enzyme reaction to calculate the AP-dependent phosphate release. FIG. 3B depicts AP-dependent phosphate release (μM) from 1 μg/μL uncapped Cas9 mRNA at 37° C.

For eGFP mRNA, 1 μg/μL of heat-denatured eGFP mRNA was subjected to 0.025 U/μL AP treatment at 4° C. and 25° C. At 2 min, 5 minutes, 10 minutes, 15 minutes, 30 minutes, 45 μminutes, and 60 minutes, reaction aliquots were diluted with water and quenched with MG quenching buffer. AP reaction was carried out in similar fashion as described above for Cas9mRNA but without the centrifugation step. Phosphate release was measured by absorbance at 620 nm. FIG. 3C depicts AP-dependent phosphate release (μM) from eGFP mRNA at 4° C., and FIG. 3D depicts AP-dependent phosphate release (μM) from eGFP mRNA at 25° C.

Across all tested temperatures, the resulting phosphate-release curves demonstrate that AP remains active and capable of removing 5′ terminal phosphates. Although reactions progress more slowly at 4° C. and 25° C. than at 37° C., overall phosphate release at later time points is greater at lower temperatures. These findings confirm that AP maintains sufficient activity over a range of temperatures suitable for capping-efficiency determination while minimizing heat-induced mRNA degradation.

### Example 5: Gradient of Capped and Uncapped mRNA

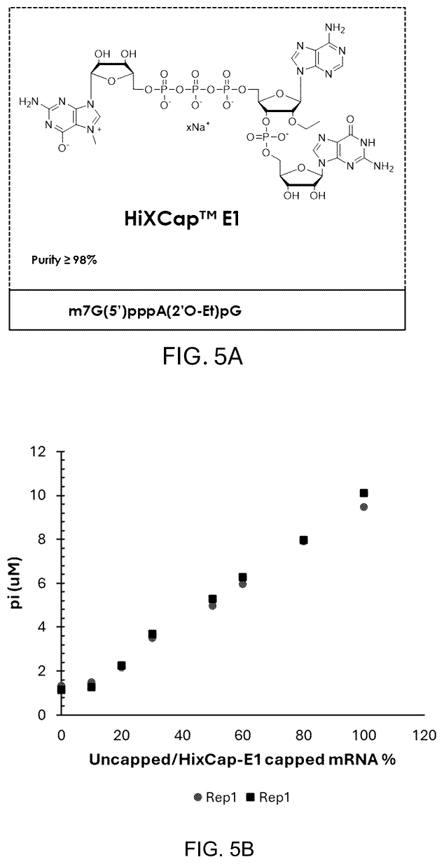

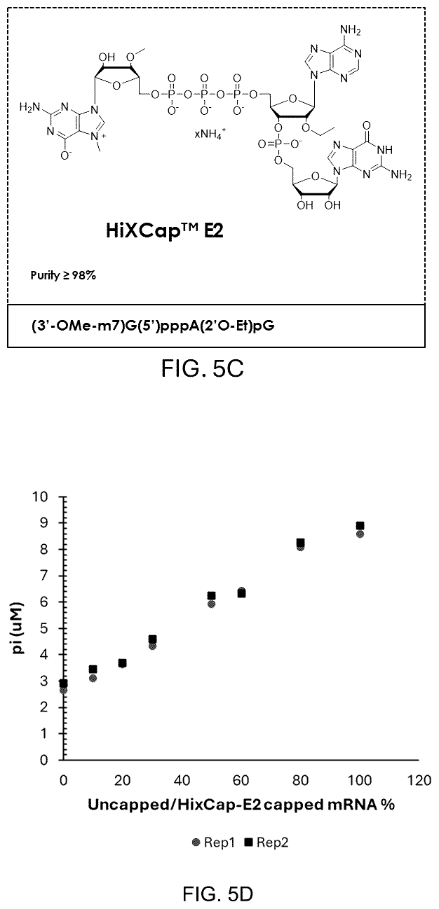

An AP digestion reaction was carried out to assess AP dependent phosphate release from modified and unmodified Fluc mRNA samples with or without a cap analog. Reaction mixtures were prepared using the following components: 0.01 U/μL RNase inhibitor, 1×AP buffer, 0.0625 U/μL TAB5 AP, and modified or unmodified 0.5 μg/μL Fluc mRNA capped with a HiXCap™ E1 or HiXCap™ E2 cap analog. The reaction volume was adjusted to 100 μL with nuclease-free water. FIG. 5A shows the chemical structure of a HiXCap™ E1 cap analog. FIG. 5C shows the chemical structure of a HiXCap™ E2 cap analog. A no-enzyme control was included to quantify AP-independent background phosphate, which was subtracted during data analysis. Reaction mixtures were incubated at 37° C. for 10 minutes, diluted 2-fold by adding 100 μL water, and immediately quenched by addition of 50 μL MG quenching solution. Samples were incubated at room temperature for 10 minutes to allow complete color development, followed by centrifugation at 15,000 g for 3 minutes to remove precipitates and clarify the supernatant.

In parallel, 80 μL of phosphate standard was combined with 20 μL of MG quenching solution in a 96-well microplate and incubated for 10 minutes at room temperature before use. For each AP-treated sample, 100 μL of the clarified, MG-quenched reaction supernatant was transferred into replicate wells of a 96-well microplate, taking care not to disturb the pellet. Optical density at 620 nm (OD₆₂₀) was measured for all samples and standards. Phosphate concentration was determined by fitting OD values to the phosphate standard curve. Enzyme-dependent phosphate release for each test sample was calculated by subtracting the phosphate detected in the no-enzyme control from the phosphate detected in the AP treated sample.

FIG. 5B is a graph depicting the quantification of AP dependent phosphate release (μM) from Fluc mRNA with or without a HiXCap™ E1 cap. FIG. 5D is a graph depicting some embodiments of quantification of AP dependent phosphate release (μM) from Fluc mRNA with or without a HiXCap™ E2 cap.

### Example 6: Alkaline Phosphatase (AP) Dependent Phosphate Release from eGFP mRNA Capped with an Anti-Reverse Cap Analog (ARCA) Cap Analog m⁷G(_{3′OMe})pppG

To evaluate capping efficiency using MG-based phosphate detection, mixtures of ARCA-capped and uncapped eGFP mRNA were prepared at defined ratios. The AP-dependent phosphate release from each mixture was quantified following AP treatment as described in Example 5.

The sample containing 100% uncapped mRNA was designated as the 100% Uncapped reference, and the phosphate release value obtained for that sample was used to normalize the uncapped percentage of all other ARCA-capped mixtures. The resulting uncapped percentage was then used to calculate capped percentage and capping efficiency.

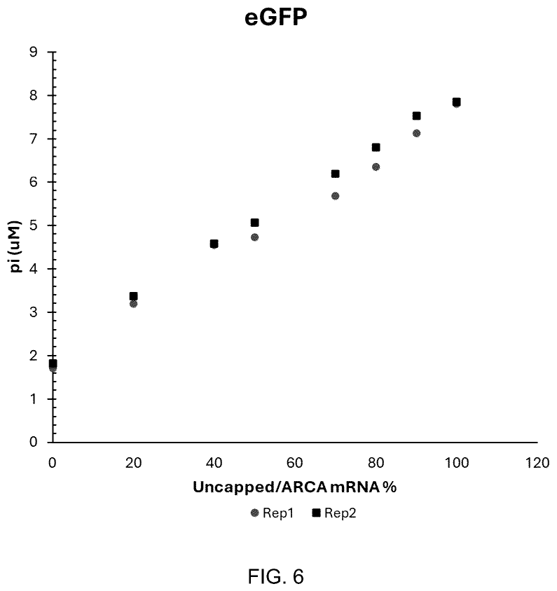

FIG. 6 is a graph showing some embodiments of quantification of AP dependent pi release from an eGFP mRNA mixture with an increasing percentage of ARCA capped mRNA.

Table 9 summarizes the tested ratios, AP-dependent phosphate (pi) release, and calculated uncapped and capped percentages.

TABLE 9

| | | AP | | | |
|---|---|---|---|---|---|
| | | dependent | | | |
| Uncapped | Capped | pi release | | | Capping |
| mRNA % | mRNA % | (μM) | Uncapped % | Capped % | efficiency |
| 0 | 100 | 1.70 | 21.8 | 78.2 | 78.2 |
| 20 | 80 | 3.19 | 40.9 | 59.1 | 73.9 |
| 40 | 60 | 4.55 | 58.3 | 41.7 | 69.5 |
| 50 | 50 | 4.73 | 60.6 | 39.4 | 78.8 |
| 70 | 30 | 5.68 | 72.9 | 27.1 | 90.4 |
| 80 | 20 | 6.36 | 81.5 | 18.5 | 92.3 |
| 90 | 10 | 7.13 | 91.4 | 8.6 | 85.8 |
| 100 | 0 | 7.80 | 100.0 | 0.0 | |

Four mixtures within the optimal quantitative window were used to calculate ARCA's capping efficiency. Normalized uncapped percentages were used to derive capped percentages according to Formula 1. The average efficiencies are presented in Table 10.

TABLE 10

| | Capped:Uncapped mRNA ratio | Rep1 | Rep2 |
|---|---|---|---|
| Ratio 1 | 0:100 | 78 | 77 |
| Ratio 2 | 10:90 | 74 | 71 |
| Ratio 3 | 20:80 | 69 | 69 |
| Ratio 4 | 50:50 | 79 | 71 |
| Average | | 75 ± 4 | 72 ± 3 |

### Example 7: Alkaline phosphatase (AP) dependent phosphate release from Cas9 mRNA capped with m⁷iGpppA(_{2′OMe})pG (GAG)

To evaluate capping efficiency of GAG capped Cas9 mRNA, mixtures of uncapped and capped Cas9 transcripts were prepared at defined ratios. Each mixture was subjected to AP treatment followed by quenching with MG phosphate detection reagent, as described in Example 5. AP-dependent phosphate release was used to quantify the relative amount of uncapped 5′ triphosphate, enabling calculation of uncapped and capped percentages.

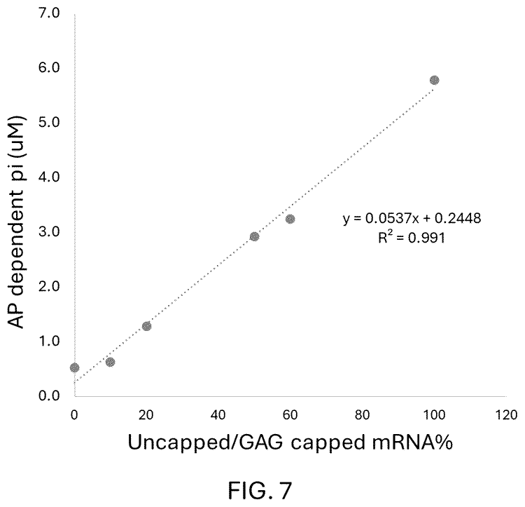

As in Example 6, the sample containing 9.0 uncapped Cas9 mRNA was designated as the 100% Uncapped reference. All other mixtures were normalized relative to this reference to determine uncapped percentage and subsequently capped percentage according to Formula 1. FIG. 7 is a graph depicting some embodiments of quantification of AP dependent phosphate (Pi) release from an uncapped/GAG capped Cas9 mRNA mixture.

Table 11 summarizes the uncapped/capped ratios tested and the resulting capped percentages.

TABLE 11

| | GAG- | AP | | | Capping |
|---|---|---|---|---|---|
| Uncapped | Capped | dependent | Uncapped | Capped | efficiency |
| mRNA % | mRNA % | Pi (μM) | % | % | % |
| 0 | 100 | 0.52 | 9.0 | 91.0 | 91.0 |
| 10 | 90 | 0.62 | 10.7 | 89.3 | 99.2 |
| 20 | 80 | 1.27 | 22.1 | 77.9 | 97.4 |
| 50 | 50 | 2.92 | 50.5 | 49.5 | 99.0 |
| 60 | 40 | 3.24 | 56.1 | 43.9 | 109.8 |
| 100 | 0 | 5.78 | 100.0 | 0.0 | |

Three mixtures (10:90, 20:80, and 50:50) within the optimal quantitative window were used to calculate Cas9 mRNA capping efficiency. The averaged results are presented in Table 12.

TABLE 12

| | Capped:Uncapped mRNA ratio | Capping efficiency % |
|---|---|---|
| Ratio 1 | 10:90 | 99.2 |
| Ratio 2 | 20:80 | 97.4 |
| Ratio 3 | 50:50 | 99.0 |
| Average | | 99 ± 1 |

### Example 8. Method Comparison

MG assays were performed as described in Example 5 using modified Fluc or Cas9 mRNA prepared with GAG, HiXCap-E1, or HiXCap-E2 cap analogs. Capping efficiency determined by the disclosed methods was compared to benchmark capping efficiencies measured using an RNaseH-LC-MS assay (Beverly et al., Anal. Bioanal. Chem. 2016). Table 13 summarizes capping efficiencies measured by both methods.

TABLE 13

| | Fluc | | | | Cas9 | |
|---|---|---|---|---|---|---|
| | U Modification | | | | | |
| | Uridine | | | N1meψ | N1meψ | |
| Cap analog | GAG | HiXCap-E1 | HiXCap-E2 | GAG | GAG | HiXCap-E2 |
| MG assay | 92 ± 3 | 93 ± 3 | 71 ± 1 | 94 ± 3 | 99 | 86 |
| RNaseH LC-MS | 99.9 | 98.3 | 95.9 | 99.8 | 99.5 | 95.5 |

The high consistency demonstrates the robust performance of the methods disclosed herein and confirms that detection of mRNA caping efficiency using the methods disclosed herein are similar to LC-MS benchmarks.

1. A method of determining mRNA capping efficiency in a test sample, comprising:
treating a test sample and one or more control samples with an alkaline phosphatase, wherein the test sample comprises capped and/or uncapped mRNAs, and the one or more control samples each comprises a known ratio of capped to uncapped mRNA;
adding a phosphate detection agent to the test sample and the one or more control samples treated with the alkaline phosphatase;
detecting the amount of free phosphate groups in the test sample and the one or more control samples;
determining the ratio of free phosphate groups in the test sample to the free phosphate groups in the one or more control samples; and
determining the mRNA capping efficiency in the test sample.

2. The method of claim 1, wherein the mRNA capping efficiency in the test sample is equal to 1 minus the ratio of free phosphate groups in the test sample to free phosphate groups in the one or more control samples.

3. The method of claim 1, wherein at least one control sample comprises 100% uncapped mRNA, at least one control sample comprises no mRNAs, and/or at least one control sample is not treated with the alkaline phosphatase.

4. (canceled)

5. The method of claim 1, wherein the capped mRNA in the test sample is prepared by in vitro transcription with a cap analog.

6. The method of claim 5, with the cap analog is selected from m⁷GpppA(_{2′-OMe})pG, m⁷GpppA(_{2′oEt})pG, m⁷GpppA(_{2′oMe})pU, m⁷GpppA(_{2′-oEt})pU, m⁷G(_{3′-OMe})pppA(_{2′-OMe})pG, m⁷G(_{3′OMe})pppA(_{2′-OMe})pU, m⁷G(_{3′-OMe})pppA(_{2′-OEt})pG, m⁷G(_{3′OMe})pppA(_{2′-OEt})pU or m⁷G(_{3′-OMe})pppG.

7. The method of claim 1, wherein the concentration alkaline phosphatase is from about 0.01 U/μL to about 5 U/μL.

8. The method of claim 1, wherein the alkaline phosphatase comprises TAB5 or CIP.

9. (canceled)

10. The method of claim 1, wherein the treating of the test sample and the one or more control samples with an alkaline phosphatase comprises incubating the test sample and the one or more control samples in the presence of the alkaline phosphatase for a period of time to allow for the release of all three 5′ terminal phosphate groups from the uncapped mRNA.

11. The method of claim 10, wherein the incubating is carried out at room temperature or at about 37° C.

12. (canceled)

13. The method of claim 10, wherein the incubating is carried out for about 10 minutes to about 1 hour.

14. (canceled)

15. The method of claim 1, wherein treating the test sample and the one or more control samples with the alkaline phosphatase is in the presence of a RNase inhibitor.

16. The method of claim 1, wherein the phosphate detection agent comprises Malachite Green.

17. The method of claim 1, wherein adding the phosphate detection agent quenches the alkaline phosphatase reaction with the test sample and the one or more control samples.

18. The method of claim 1, further comprising centrifuging the test sample and the one or more control samples after adding the phosphate detection agent.

19. The method of claim 1, wherein detecting the amount of free phosphate groups in the test sample and the one or more control samples comprises measuring the optical density of the test sample and the one or more control samples,
wherein the optical density of the test sample and the one or more control samples is measured at about 600 nm to about 660 nm, or about 620 nm.

20. (canceled)

21. The method of claim 1, wherein the amount of mRNA in the test sample is from about 0.5 μg/μL to about 10 μg/μL.

22. The method of claim 1, wherein the amount of free phosphate groups detectable by the phosphate detection agent is from about 0.01 to about 100 μM, or from about 0.02 μM to about 40 μM.

23. (canceled)

24. (canceled)

25. The method of claim 1, wherein the capping efficiency of the test sample determined by the method is within 10% of the caping efficiency of the test sample as measured by LC-MS.

26. The method of claim 1, further comprising heating the test sample and one or more control samples prior to treating the test sample and one or more control samples with an alkaline phosphatase.

27. The method of claim 26, wherein the test sample and one or more control samples are heated to between about 65-95° C.

28. The method of claim 26, wherein the test sample and one or more control samples are heated for between about 2-10 minutes.

29. (canceled)
